(19) United States
(12) Patent Application Publication
(10) Pub. No.: US 2024/0122761 A1
(43) Pub. Date: Apr. 18, 2024

(54) MULTI-PIECE EYE MASK

(71) Applicant: MANTA SLEEP, LLC., JACKSON, WY (US)

(72) Inventors: BENJAMIN SCHWARZ, JACKSON, WY (US); MARK ZHANG, JACKSON, WY (US)

(21) Appl. No.: 18/047,131

(22) Filed: Oct. 17, 2022

(51) Int. Cl. A61F 9/04; H04R 5/033

(52) U.S. Cl. CPC A61F 9/04; H04R 5/033

(57) ABSTRACT

A multi-piece eye mask is provided and has a first headband, a second headband, and two speaker assemblies. The first headband is configured to cover the user's eyes and ears. The second headband is detachably mounted on the first headband. The speaker assemblies are mounted in the second headband and corresponding to the user's ears in location. The first headband can be separated from the second headband, which contains the eye cups, and the first headband with the eye cups is independently washable.

### BACKGROUND OF THE PRESENT DISCLOSURE

### 1. Field of the Present Disclosure

The present disclosure relates to an article for daily use, especially to an eye mask that shades light.

### 2. Description of the Prior Arts

An eye mask can be a daily necessity for helping people to sleep. If the brightness of the environment is high, it can affect the sleep quality and even make many people struggle to fall asleep. Therefore, an eye mask worn during sleep for shading light from the environment can become an important apparatus to maintain or improve sleep quality.

An eye mask may have two surfaces, one of the surfaces is configured to contact the user's skin, and the other is configured to shade the light. The surface configured to contact skin may be tender and washable, but the surface configured to shade the light may become fragile during washing or form a pattern that will fade after washing. Often, people are thus resistant to washing the eye mask.

However, people often sweat in sleep, so the eye mask that contacts the skin may absorb sweat continuously. After a period of time of use, the user may feel itchy or uncomfortable when wearing the eye mask and then have to wash it.

Environmental noise also bothers people during sleep. Thus, many people like to sleep with music or white noise, which blocks the noise from the environment and provides another way to help them fall asleep. As a result, some eye masks include one or more speakers to cover the user's ear. The speakers are made of electric components, so the eye mask with one or more speakers is generally not washable. If the user cannot tolerate an uncomfortable eye mask, but is resistant or cannot clean it, a new eye mask is needed but this is expensive. Accordingly, a need remains for improved sleep masks having speakers.

### SUMMARY OF THE PRESENT DISCLOSURE

The present disclosure provides for a multi-piece eye mask that is washable and can further include one or more speakers.

In an example, a multi-piece eye mask is provided having a first headband, a second headband, and at least two speaker assemblies. The first headband is configured to cover the user's eyes and ears. The second headband is detachably mounted on the first headband. The speaker assemblies are mounted in the second headband and corresponding to the user's ears in location.

The first headband is configured to cover the user's eyes and ears and thus may contact the user's skin and absorb sweat. The first headband and the second headband can be separated and then leaving the second headband behind, the first headband can be configured to be washable. With the multi-piece eye mask of the present disclosure, the user can wash a dirty portion of the eye mask (i.e., the first headband that does not include the speakers) but still keep the speaker assemblies intact within the second headband.

Other objectives, advantages and novel features of the present disclosure will become more apparent from the following detailed description when taken in conjunction with the accompanying drawings.

### BRIEF DESCRIPTION OF THE DRAWINGS

FIG. 1 is a front view of a multi-piece eye mask in accordance with the present disclosure;

FIG. 2 is a rear view of the multi-piece eye mask of FIG. 1;

FIG. 3 is a top view of the multi-piece eye mask of FIG. 1;

FIG. 4 is a front view of a first headband of the multi-piece eye mask of FIG. 1;

FIG. 5 is a rear view of the first headband of the multi-piece eye mask of FIG. 4;

FIG. 6 is a front view of a second headband of the multi-piece eye mask of FIG. 1;

FIG. 7 is a rear view of the second headband of the multi-piece eye mask of FIG. 6;

FIG. 8 is a front view of an eye cup of the multi-piece eye mask of FIG. 2;

FIG. 9 is a rear view of the eye cup of the multi-piece eye mask of FIG. 8;

FIG. 10 is a perspective view of a multi-piece eye mask in accordance with another embodiment of the present disclosure; and

FIG. 11 is an exploded view of the multi-piece eye mask of FIG. 10.

### DETAILED DESCRIPTION OF THE PREFERRED EMBODIMENTS

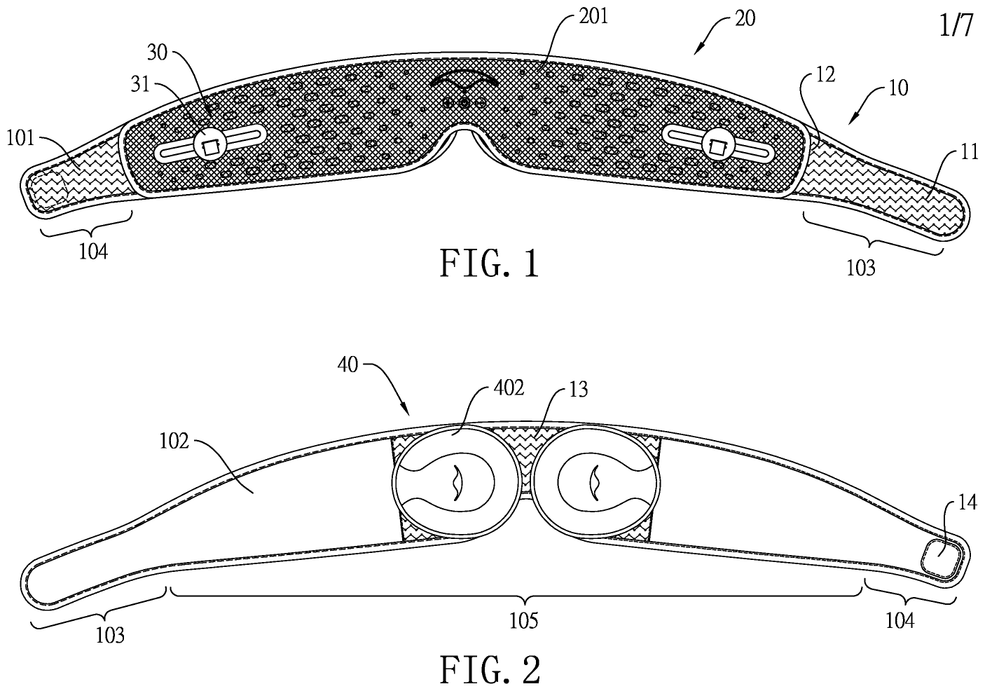

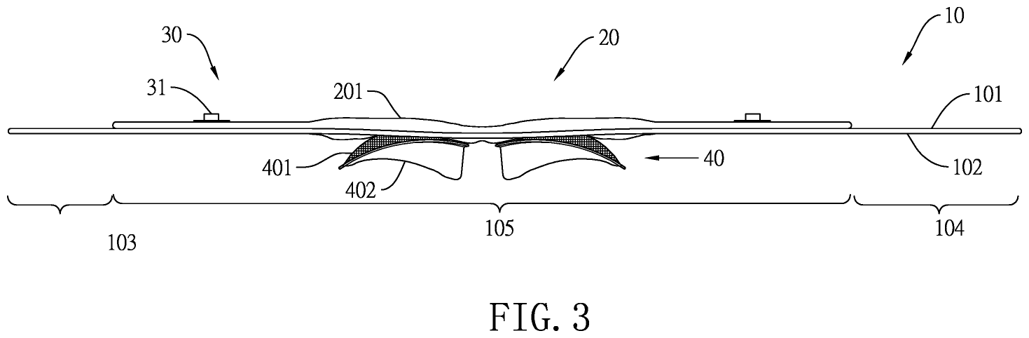

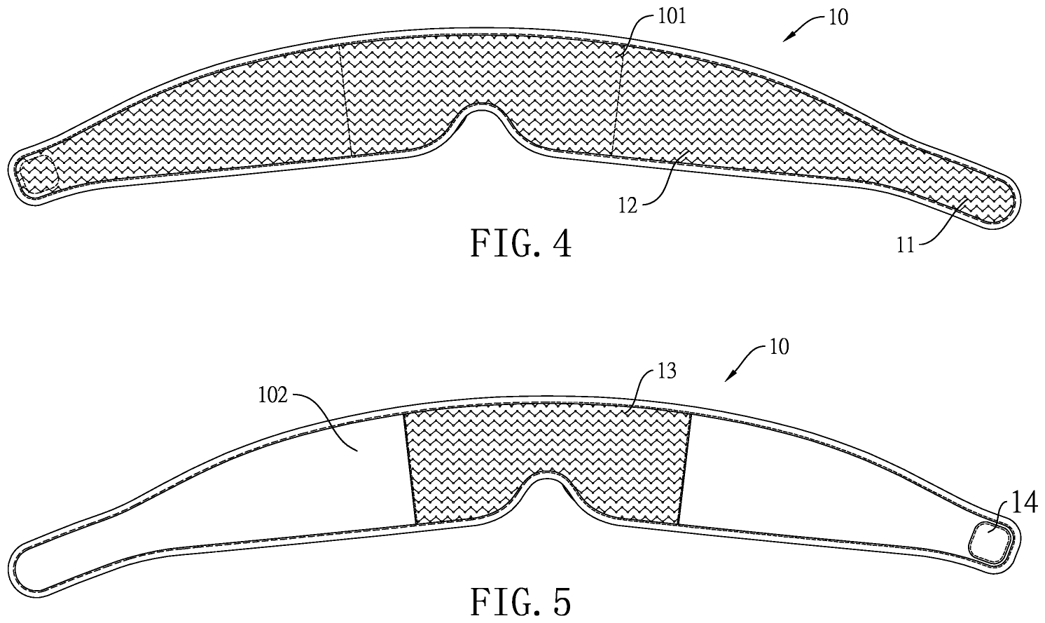

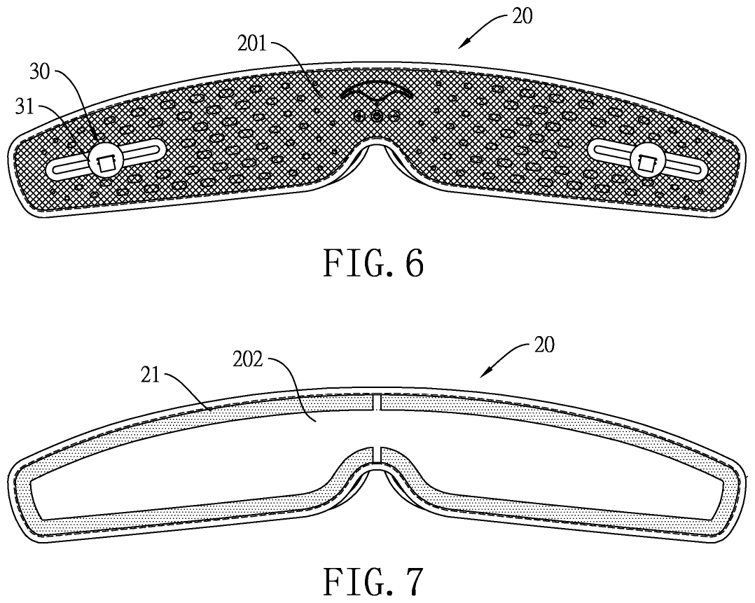

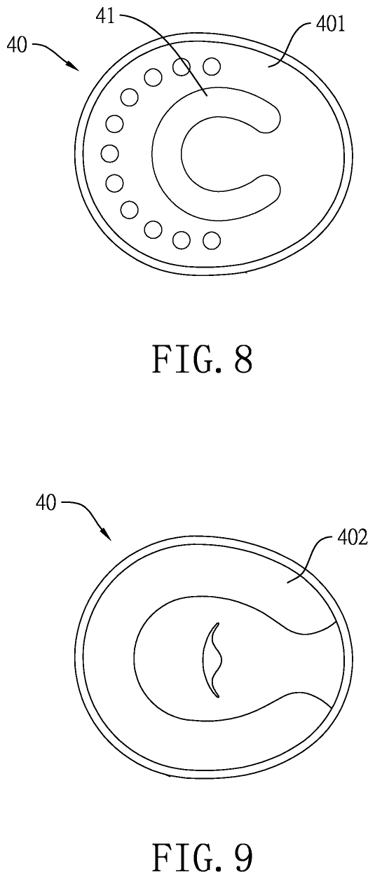

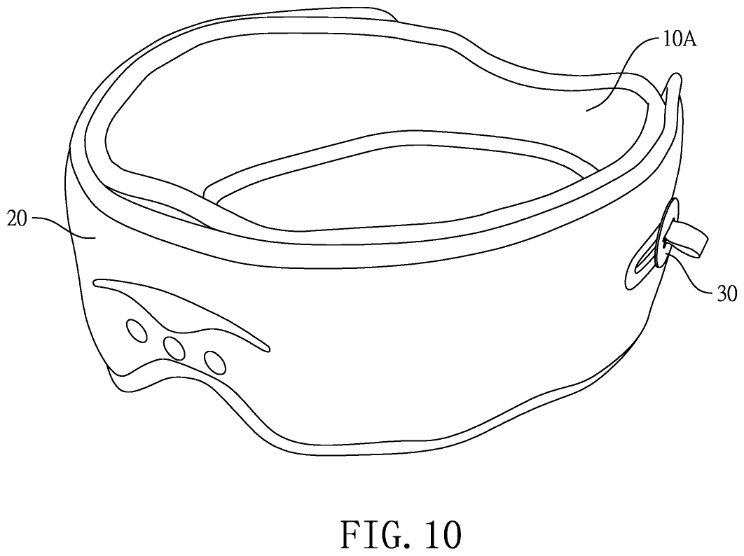

Referring to FIG. 1 to FIG. 9, a multi-piece eye mask is provided in accordance with the present disclosure which includes a first headband 10 and a second headband 20. The multi-piece eye mask may selectively include at least one speaker assembly 30 and/or two eye cups 40. In an example, the multi-piece eye mask includes two speaker assemblies 30 and two eye cups 40, but it is not limited thereto.

The first headband 10 is configured to cover a user's eyes and ears. The first headband 10 can be made of fabric, so the first headband 10 is comfortable and breathable and is capable of absorbing sweat. Moreover, the first headband 10 is configured to be washable. According to the present disclosure, being washable means that the first headband 10 will generally stay intact under standard washing conditions which may include being washed in a laundry machine or being hand washed. In this example, the first headband 10 does not include any electronic components which could be damaged or destroyed if exposed to excessive water or normal washing/laundry conditions. Moreover, the first headband 10 can include an internal foam material covered by fabric to provide added comfort. The internal foam material should also be configured to be washable.

The first headband 10 defines a front surface 101 and a rear surface 102, a first end 103 and an opposite second end 104, and a middle section 105 between the first end 103 and the second end 104. The front surface 101 and the rear surface 102 are opposite each other. The rear surface 102 is configured to cover a user's eyes and ears. The first end 103 and the second end 104 are opposite two ends of the first headband 10. The first headband 10 can be curved and the middle section 105 can be configured to be higher than the first end 103 and the second end 104 when worn by the user, and thereby the user may have better wearing experience. In this embodiment, the first end 103 is longer than the second end 104.

In an example, the first headband 10 includes an end fluff layer 11, a front fluff layer 12, and a rear fluff layer 13. The end fluff layer 11 is formed on the front surface 101 of the first end 103. The front fluff layer 12 is formed on the front surface 101 of the middle section 105. The end fluff layer 11 and the front fluff layer 12 may be connected to each other and formed integrally. The rear fluff layer 13 is formed on the rear surface 102 of the middle section 105. In this example, rear fluff layer 13 extends only a portion of rear surface 102 and corresponds to an area designed to cover the eyes of a user or to host eye cups 40.

The first headband 10 includes an attach pad 14. The attach pad 14 is provided on the rear surface 102 of the second end 104. The attach pad 14 is configured to detachably connect to the end fluff layer 11, such that the second end 104 is secured on the first end 103, thereby the first headband 10 forming a loop. Thus, a user's head may be enveloped in the first headband 10 and secured in place for using during sleep and centering the eye cups 40 or the middle section 105 substantially around the eyes of the user. Attach pad 14 can include an adhesive surface for detachably securing the second end 104. An adhesive surface may include a fastener like a hook-and-loop fastener, commonly referred to as VELCRO. Each end can be include a corresponding fastener strip that can be attached via any attachment means like glue, sowing, or otherwise.

The second headband 20 is configured to be detachably mount on the first headband 10, and the two speaker assemblies 30 are provided and secured in the second headband 20 and correspond to the user's ears in location. Typically, the second headband 20 is not washable since it contains electronic components (e.g., the speaker assemblies 30).

In this example, the second headband 20 defines at least two inner spaces positioned opposite from each other and at least one slot. The two inner spaces correspond to the user's ears in location and each forms a pocket or a cavity for receiving a speaker assembly. Each one of the at least one slot is formed to allow access to at least one of the inner spaces. In this embodiment, the second headband 20 defines two slots and each slot communicates with a respective one of the corresponding inner spaces. Each slot can be formed to extend horizontally along a horizontal axis generally defined by the sleep mask when fully extended. This defines a length of the slot and a width is defined perpendicular to the axis of the length of the slot. The width of each slot is sized and shaped to be smaller than a width defined by the corresponding inner space

The two speaker assemblies 30 are provided in the inner spaces, respectively. At least one of the speaker assemblies 30 is configured to be manually moved or adjusted within in the corresponding inner space. In an example, one of the speaker assemblies 30 is adjustable in location, but the other one may not.

The speaker assemblies 30 can be configured to be coupled to a hidden control unit (not shown in the drawings) having multiple buttons, e.g. a power-on button, connect button, and volume control buttons. The speaker assemblies 30 can be coupled to the control unit which is coupled to a power supply like a battery. In this example, the power supply is a rechargeable battery that further includes an effectively concealed charging port. The speaker assemblies 30 are further coupled to the control unit through hidden wires concealed within the first headband. It is further noted that the control unit and/or the speaker assemblies are wirelessly accessible to allow the playing of sound or music from a wireless source like a music or noise app of a smartphone, tablet or otherwise. A wireless communication protocol or module can be incorporated into the control unit to allow for BLUETOOTH compatibility or otherwise.

In an example, in use, the user may adjust the entire eye mask so that the speaker assembly 30 that cannot be adjusted to align or near one ear and then adjust the movable speaker assembly 30 with respect to the entire eye mask to align or near the other ear. In another embodiment, the speaker assemblies 30 are movable and adjustable within the corresponding inner spaces.

In a further example, the speaker assemblies 30 each includes a speaker unit and an operation unit 31. The speaker unit is configured to be loose or floating within the pocket formed by the inner space and movably positioned within one of the inner spaces. A general size of the speaker unit is larger than that of the at least one slot, such that the speaker unit may not easily be removed from or unintentionally fall out of the inner space.

The operation unit 31 is connected to the speaker unit via the at least one slot, which allows the user to manually adjust or manipulate the operation unit to slide or move the connected speaker unit 30. In this embodiment, the operation unit 31 includes a tab and selectively has a positioning piece. The tab has two ends. One of the ends of the tab is connected to the speaker unit and the other end extends out of from the slot. The positioning piece is mounted on the tab and located between the two ends of the tab. A width of the positioning piece is larger than that of the at least one of the slot.

The second headband 20 includes a front surface 201 and a rear surface 202. The rear surface 202 of the second headband 20 faces the user and includes an attaching portion 21. The attaching portion 21 is provided on the rear surface 202 of the second headband 20 and configured to detachably attach onto the front fluff layer 12 of the first headband 10, such that the second headband 20 is detachably secured on the first headband 10. The attaching portion 21 may be composed of at least one attaching strip, and the attaching strip is provided on the second headband 20 and extends along a contour of the second headband 20. The attachment portion may include an adhesive surface like a fastener like a hook-and-loop fastener, commonly referred to as VELCRO. Each end can include a corresponding fastener strip that can be attached via any attachment means like glue, sowing, or otherwise.

The two eye cups 40 can be detachably mounted on the first headband 10 and configured to cover the user's eyes respectively. Each one of the eye cups 40 has a front side 401 and a rear side 402 opposite each other. The rear side 402 is configured to cover one of the user's eyes. Each one of the eye cups 40 comprises an attaching portion 41. The attaching portion 41 of the eye cup 40 is mounted on the front side 401 of the eye cup 40 and configured to detachably attach onto the rear fluff layer 13 of the first headband 10, such that the eye cup 40 is secured on the first headband 10. The eye cups 40 can be made of fabric, so the eye cups 40 are configured to be comfortable and breathable and are suitable for absorbing at least some of the user's sweat. Moreover, the eye cups 40 can be configured to be washable.

In another embodiment, the multi-piece eye mask may not have eye cups 40, so the user's eyes are shaded by the first headband 10.

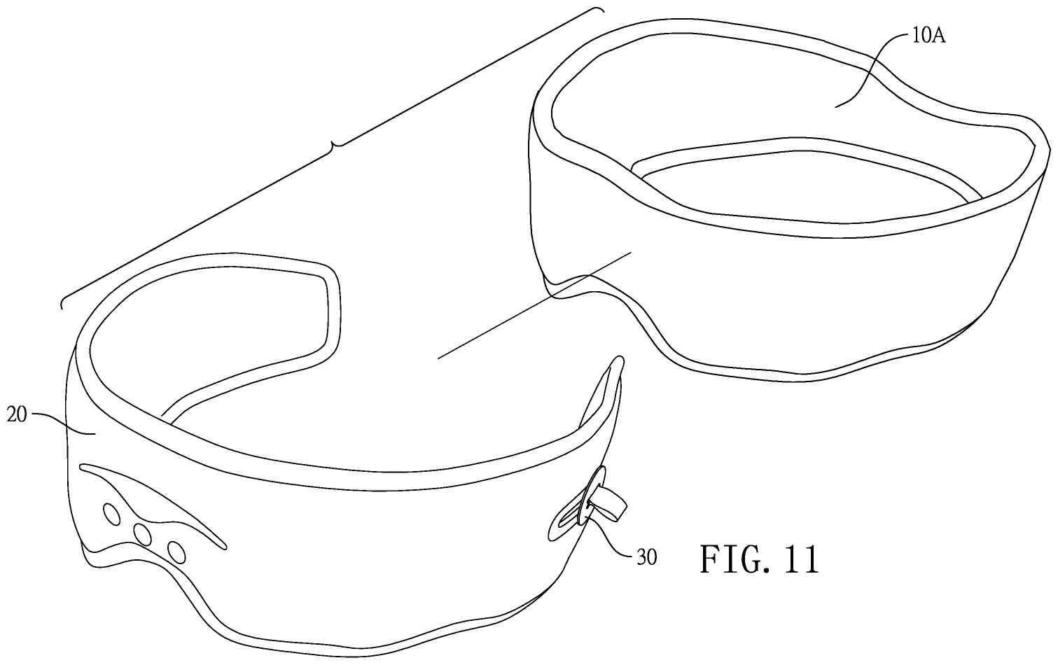

Please refer to FIG. 10 and FIG. 11. A multi-piece eye mask in accordance with another embodiment of the present disclosure is provided. In this embodiment, the first headband 10A is a loop such that the first headband 10A may be secured on the user's head without any attach pad, which makes the entire first headband 10A tender and provides the user better experience. The loop-shaped first headband 10A is washable and breathable and is capable of absorbing sweat. Besides, the entire first headband 10A may be made of elastic material, an elastic ribbon for example, or only a section of the first headband 10A is made of elastic material. The multi-piece eye mask may still has a second headband 20 detachably mounted on the first headband 10A and two speaker assemblies 30 mounted in the second headband 20. When wearing the multi-piece eye mask, the user can envelope head in the first headband 10A arbitrarily, and then attach the second headband 20 on the first headband 10A according to the position of eyes and ears. Or, the user's head can be enveloped by both the first headband 10A and the second headband 20, and then adjust the first headband 10A until the second headband 20 aligning the position of eyes and ears.

With the aforementioned structures, the multi-piece eye mask of the present disclosure includes a first headband 10 and the eye cups 40, and the first headband 10 and the eye cups 40 are configured to contact the user. Therefore, after putting on the present multi-piece eye mask, the user may separate the first headband 10, the second headband 20, and the eye cups 40, and then only wash the first headband 10 and the eye cups 40 that are no longer clean or have absorbed the user's sweat. As a result, with the multi-piece eye mask of the present disclosure, the user can wash the dirty part of the eye mask but still keep the speaker assemblies 30 intact.

Even though numerous characteristics and advantages of the present disclosure have been set forth in the foregoing description, together with details of the structure and features of the present disclosure, the disclosure is illustrative only. Changes may be made in the details, especially in matters of shape, size, and arrangement of parts within the principles of the present disclosure to the full extent indicated by the broad general meaning of the terms in which the appended claims are expressed.

What is claimed is:

1. A multi-piece eye mask comprising:
a first headband configured to cover eyes of a user; and
a second headband detachably mounted on the first headband.

2. The multi-piece eye mask as claimed in claim 1, wherein:
the first headband configured to cover ears of the user; and
the multi-piece eye mask further comprises:
at least one speaker assembly mounted in the second headband.

3. The multi-piece eye mask as claimed in claim 2, wherein the at least one speaker assembly corresponds to one of the user's ears in location.

4. The multi-piece eye mask as claimed in claim 2, wherein the at least one speaker assembly includes two said speaker assemblies.

5. The multi-piece eye mask as claimed in claim 4, wherein each one of the speaker assemblies corresponds to a respective one of the user's ears in location.

6. The multi-piece eye mask as claimed in claim 1, wherein the first headband is made of a washable fabric.

7. The multi-piece eye mask as claimed in claim 1 further comprising two eye cups positioned on the first headband and configured to cover the user's eyes respectively; wherein the two eye cups are washable.

8. The multi-piece eye mask as claimed in claim 7, wherein the two eye cups are removably positioned on the first headband.

9. The multi-piece eye mask as claimed in claim 7, wherein the two eye cups are made of fabric.

10. The multi-piece eye mask as claimed in claim 1, wherein the first headband has a first end, a second end, and a middle section, the first end and the second end are opposite two ends of the first headband, the middle section is between the first end and the second end, and the middle section is higher than the first end and the second end in location.

11. The multi-piece eye mask as claimed in claim 1, wherein:
the first headband has a first end and a second end, the first end and the second end are opposite two ends of the first headband, the first end has a front surface, the second end has a rear surface, the front surface of the first end and the rear surface of the second end face inverse directions;
a fluff layer is formed on the front surface of the first end; and
an attach pad is mounted on the rear surface face of the second end and configured to detachably attach onto the fluff layer, such that the second end is secured on the first end.

12. The multi-piece eye mask as claimed in claim 11, wherein the first end is longer than the second end.

13. The multi-piece eye mask as claimed in claim 1, wherein:
the first headband has a front surface and a rear surface opposite each other, and the rear surface faces the user; the first headband comprises:
a fluff layer formed on the front surface;
the second headband has a rear surface facing the user and comprises:
an attaching portion mounted on the rear surface of the second headband and configured to detachably attach onto the fluff layer, such that the second headband is detachably secured on the first headband.

14. The multi-piece eye mask as claimed in claim 13, wherein the attaching portion extends along a contour of the second headband.

15. The multi-piece eye mask as claimed in claim 8, wherein:
the first headband has a front surface and a rear surface opposite each other, and the rear surface faces the user; the first headband comprises:
a fluff layer formed on the rear surface;
each one of the eye cups has a front side and a rear side, the rear side is configured to cover one of the use's eyes; each one of the eye cups comprises:
an attaching portion mounted on the front side of the eye cup and configured to detachably attach onto the fluff layer, such that the eye cup is detachably secured on the first headband.

16. The multi-piece eye mask as claimed in claim 1, wherein the first headband is a strip or a closed loop.
